(19) United States
(12) Patent Application Publication
(10) Pub. No.: US 2005/0182393 A1
(43) Pub. Date: Aug. 18, 2005

(54) Multi-energy ablation station

(75) Inventors: Marwan Abboud, Pierrefonds (CA); Sean Carroll, Beaconsfield (CA); Fred Milder, Brookline, MA (US); Jean-Pierre Desmarais, Saint-Lazare (CA); Steve Arless, Beaconsfield (CA); George Klein, London (CA)

Correspondence Address: Christopher & Weisberg, P.A.;Suite 2040, 200 East Las Olas Boulevard, Fort Lauderdale, FL, 33301, US

(73) Assignee: CryoCath Technologies Inc.

(21) Appl. No.: 11/106,839

(22) Filed: Apr. 15, 2005

Related U.S. Application Data
(62) Division of application No. 10/364,764, filed on Feb. 11, 2003.

(51) Int. Cl. A61B 18/02; A61B 18/18

(52) U.S. Cl. 606/20; 606/21; 606/34; 606/41

(57) ABSTRACT

An integrated multiple energy ablation system that allows for a variety of ablation procedures to be performed without the interchanging of catheters. A console is provided that is connected to one or more energy treatment devices such as catheters or probes, via an energy-delivering umbilical system. A processor in the console allows a user to selectively control which type of energy is released into the umbilical system and delivered to the energy treatment devices. Cryogenic fluid, RF energy, microwave or direct current as well as laser energy can be supplied in order to cover a wide range of ablation techniques. The integrated ablation station is designed to be compatible with commercial catheters and allows for sequential or simultaneous ablation and mapping procedures to be performed when a deeper and wider lesion capability and/or a broader temperature ablation spectrum is desired.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

The present invention relates to a method and system for performing various methods of ablation and more specifically to a multiple-energy fully integrated ablation system fully compatible with existing cryocatheters and RF catheters and that allows for selected individual or sequential ablation techniques, such as cryoablation, RF ablation or cold-tip RF ablation to be performed without the need to switch and interchange catheters.

### BACKGROUND OF THE INVENTION

The present invention relates to a multiple-energy ablation system that allows for various methods of ablation without the need to interchange catheters. Many medical procedures are performed using minimally invasive surgical techniques, wherein one or more slender implements are inserted through one or more small incisions into a patient's body. With respect to ablation, the surgical implement can include a rigid or flexible structure having an ablation device at or near its distal end that is placed adjacent to the tissue to be ablated. Radio frequency energy, microwave energy, laser energy, extreme heat, and extreme cold can be provided by the ablation device to kill the tissue.

The use of fluids with low operating temperatures, or cryogens, has begun to be explored in the medical and surgical field. Of particular interest are the potential use of catheter based devices, which employ the flow of cryogenic working fluids therein, to selectively freeze, or “cold-treat”, targeted tissues within the body. Catheter based devices are desirable for various medical and surgical applications in that they are relatively non-invasive and allow for precise treatment of localized discrete tissues that are otherwise inaccessible. Catheters may be easily inserted and navigated through the blood vessels and arteries, allowing non-invasive access to areas of the body with relatively little trauma.

Catheter-based ablation systems are well known in the art. A cryogenic device uses the energy transfer derived from thermodynamic changes occurring in the flow of a cryogen therethrough to create a net transfer of heat flow from the target tissue to the device, typically achieved by cooling a portion of the device to very low temperature through conductive and convective heat transfer between the cryogen and target tissue. The quality and magnitude of heat transfer is regulated by the device configuration and control of the cryogen flow regime within the device.

Cryomapping is a procedure that chills conducting target tissue to create a transient electrical effect. By temporarily chilling the target tissue, it allows for precise site confirmation in order to prevent inadvertent ablation. Cryoadhesion is another procedure that occurs during cryomapping to ensure the catheter tip remains at the target cite for a seamless transition to cryoablation. In a cryoadhesion procedure, the tip of the catheter firmly attaches to the tissue when it freezes thereby reducing the risk of accidental slippage of the catheter tip.

Treatment of cardiac arrhythmias through selective ablation of cardiac tissue may be improved if, prior to ablation, the local electrical activity of the region can be suppressed to determine the effectiveness of the proposed lesion site in stopping the arrhythmia. Localized electrical activity may be suppressed by chilling small regions of myocardial tissue and then performing electrocardiographic mapping to evaluate the arrhythmia. This technique of cooling and mapping is called “zero-degree”, “ice”, or “cryo” mapping. If the proposed lesion site would be effective, as determined by the ice mapping, to eliminate the arrhythmia, the site is ablated.

Radio Frequency (RF)-based ablation systems are also well known in the art. In RF ablation procedures, a specially designed probe is directed into a patient's target region. Once the physician has performed a diagnostic electrophysiology (EP) study, he would insert another ablation catheter that is designed to deliver radio frequency energy to a specific focus within the patient's heart. Most ablation catheters are quadrapolar with a larger distal tip that contains a mechanism that delivers the RF energy to the heart. As this energy passes through the tissue, impedance to the signal causes heat which destroys the cells within a 2 mm range of the catheter tip.

As with the diagnostic catheters, there are many different types of ablation catheters. These various types of catheters are designed to aide the physician in ablating different locations in patients of variable size. Many physicians choose one or two ablation catheters that they prefer to use, although a difficult case may cause the doctor to switch to other styles.

The ablation catheter delivers radio frequency energy to the heart to destroy cells that may be causing the patient's arrhythmia. This is achieved using an RF generator. Energy from the generator is sent through a connecting cable to the ablation catheter where it is focused on a specific site within the patient's heart. The goal is to form a small, discreet scar at the selected site. Once formed, the scar prevents the transmission of electrical signals through that region and hopefully, terminates the arrhythmia.

Most of the RF generators available today are very similar. With the exception of some minor differences, they all perform the same functions. Each unit generates radio frequency energy that is sent on to the catheter. Each also displays the temperature at the tip of the catheter, the power required to achieve that temperature, the impedance measured by the system and the amount of time the ablator has been delivering RF energy.

When the ablation system is set up, either the temperature or the power control must be selected. There is little operational difference between which one is chosen, although temperature control appears to be used most of the time. A desired temperature is selected and programmed into the generator. The maximum power and duration of individual ablation runs, also called “burns”, are chosen. When these parameters are all entered, the ablation process is ready to begin.

Once the ablation catheter is activated, continuous readings of power display, temperature, impedance and time are displayed. As the ablation run progresses, the physician must be informed of the afore-mentioned values frequently, such as for example, at least every 15 seconds. If there is a significant change in one of the parameters, the technician must let the physician know immediately to avoid any serious complications during the ablation. A sudden increase in temperature may indicate the catheter is not in proper contact with the cardiac tissue and the blood in the immediate region could become too hot. A similar increase in impedance may indicate that the catheter has advanced and may perforate the myocardium. If the impedance increases too quickly, most units have an automatic cutoff that will disengage the ablation device.

As with cryoablation, RF ablation offers many advantages over open surgical procedures. Patients are often unable to be treated with conventional surgical techniques. RF ablation can be performed multiple times on different occasions. It is often debilitating when a patient must undergo a second or larger surgical procedure.

Yet another type of ablation procedure is what is known in the art as “cooled-tip” or “cold-tip” RF ablation. The Cooled tip RF Ablation Catheter is a minimally invasive device designed to ablate large area of tissue using radiofrequency energy. In radiofrequency ablation, excessive heating of the tissue and the ablation electrode often limits the level of energy delivered and therefore the success of the treatment. Further, a coolant can be disposed in a chamber in communication with the catheter to help in dissipating heat created by the electrodes. This is useful to minimize the potential damage such as charring that may occur when the RF power is increased to create greater lesions. Incorporating a closed system of fluid circulation allows circulating fluid to cool the catheter ablation electrode during delivery of radiofrequency energy. An automatic controller system injects fluid into a catheter lumen that circulates the fluid to the tip electrode and back to the controller. The circulation of fluid draws heat away from the metal electrode and from the electrode-to-tissue interface, which will allow the delivery of higher radiofrequency energy power levels without excessive heating. Higher power levels allow for creation of wider and deeper lesions than those created with lower power levels, increasing the likelihood of a successful ablation.

While all of these methods of ablation have their advantages, there may be certain circumstances where one method is desired over the other. Further, these circumstances may change rapidly, requiring that a cryocatheter be quickly replaced by an RF catheter and vice-versa. For example, when a linear lesion or a hazardous focal lesion is required, cryoablation is preferred. However, when a deeper lesion is required, such as to treat atrial flutter, RF ablation or a combination RF/Cryoablation procedure is required. Further, there may be the need for simultaneous use of multiple catheters of varying types. There is presently no system available that can provide a medical technician with the freedom to select an ablation technique based on the patient's medical condition, and perform sequential or simultaneous ablation procedures without the difficulty and time-consuming effort of constantly replacing one type of catheter with another.

Accordingly, given the different types of existing ablation procedures, it would be desirable to provide an ablation station that allows for the sequential or simultaneous use of various types of ablation procedures and that is adaptable and compatible with all types of existing ablation catheters. For example, cryomapping of a specific target tissue area may be followed by an RF ablation, a cryoablation, or a cooled-tip ablation procedure.

It is also desirable to provide an integrated ablation system that provides deeper and wider lesion capability or selective and narrower lesion capability when it operates near a sensitive area such as the Atrioventicular (AV) node.

It would also be desirable to provide an ablation system that, in addition to supplying the necessary cryogenic fluid in order to perform safe and effective cryoablation procedures, adds a radio frequency delivery system to provide the medical technician with a broader temperature ablation spectrum.

Such systems would be able to control and maintain the tip temperature of the catheter between cryoablation temperatures and +100 degrees Celsius, for example, which allows the user to use cryoablation, cryomapping, cryoadhesion, cooled-tip RF and RF-only ablation techniques, or to apply any combination of these procedures in any sequence or in any co-temporal configuration.

### SUMMARY OF THE INVENTION

The present invention advantageously provides an integrated ablation station having multiple energy treatment capabilities for performing sequential or simultaneous ablation techniques to a target tissue area. The ablation station provides a delivery of healing energy and provides a means to deliver to and/or remove heat from the tissue.

According to one aspect of the invention, the ablation station comprises a treatment energy generation station capable of supplying one or more different forms of treatment energy to one or more energy treatment devices, such as catheters, and an umbilical system having a first end coupled to the mating end of the one or more energy treatment devices and a second end coupled to the energy generation station. The treatment energy generation station comprises a processor that can selectively control the dispersement of the one or more different forms of treatment energy and selectively activate and control the one or more energy treatment devices.

According to another aspect of the invention, a method of applying treatment energy to a target tissue area using multiple ablation techniques is provided. The method comprises the steps of first providing a treatment energy generation station capable of supplying one or more different forms of treatment energy to one or more energy treatment devices, which are coupled to the treatment energy generation station. Treatment energy is then selectively supplied to the selected one or more energy treatment devices and the target tissue area is ablated using the selected one or more energy treatment devices.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a diagrammatic depiction of an embodiment of the multi-energy ablation station of the present invention utilizing one catheter.

FIGS. 2A-2D illustrates typical ablation profiles utilizing the present invention.

FIG. 3 is a diagrammatic depiction of the present invention showing the adaptability of the multi-energy ablation station of the present invention utilizing one of a variety of energy generation stations.

FIG. 4 is a diagrammatic depiction of the present invention with a self-contained RF generator located within the console.

FIG. 5 is a diagrammatic depiction of the present invention, with multiple-energy console coupled to one of a number of different catheters.

### DETAILED DESCRIPTION OF THE INVENTION

The present invention provides an ablation station that allows for the selective control and utilization of catheters to perform a variety of ablation techniques. While cryomapping, RF ablation, cryoadhesion, cryoablation, and cold tipped RF ablation techniques are all useful, it becomes cumbersome and costly to remove and replace existing catheters in order to effectively ablate a tissue region with a different form of ablation. The ablation system of the present invention provides a unique way to interact with existing catheters, of all designs, in order to effectively treat tissue treatment regions.

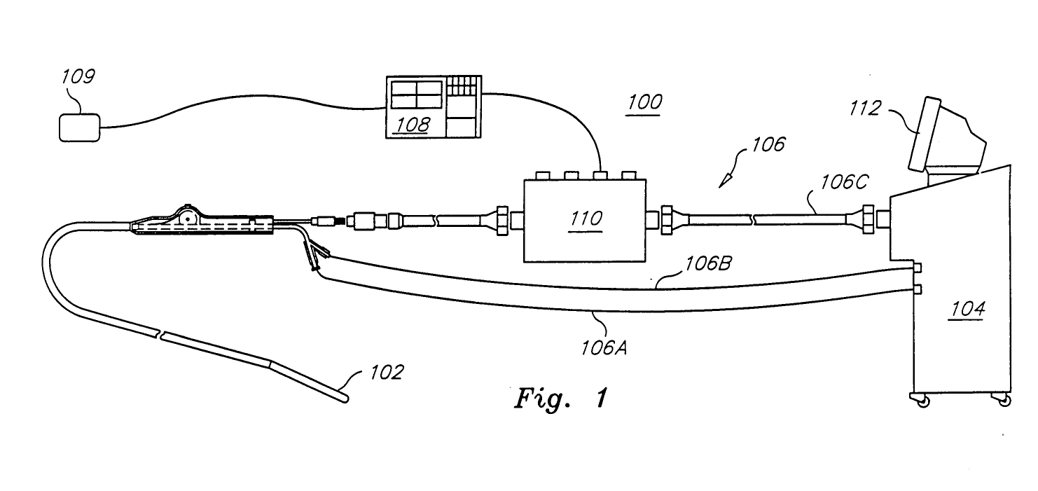

FIG. 1 illustrates one embodiment of the present invention. In this embodiment, a combination multi-energy cryoablation/radiofrequency ablation system 100 of the present invention is shown. The system includes a console 104 coupled to one end of an umbilical system 106. The opposing end of umbilical system 106 is coupled to an energy treatment device 102. Energy treatment device may be a medical probe, a catheter, a balloon-catheter, as well as other devices commonly known in the art that are smooth enough to pass easily through blood vessels and heart valves. Umbilical system 106 includes and electrical umbilical 106C that contains signal lines for monitoring and/or mapping tissue and cardiac regions and can be ultimately coupled to an ECG monitor. A cooling injection umbilical 106A and a vacuum umbilical 106B also comprise umbilical system 106. Cooling injection umbilical 106A and vacuum umbilical 106B provide respective inlet and return paths for a refrigerant or coolant used to cool a tissue-treating end of the catheter 102.

The console 104 provides the user interface to the system and houses the electronics and software for controlling and recording the ablation procedure, controlling the delivery of the liquid refrigerant under pressure through the umbilical to the catheter, controlling the recovery of the expanded refrigerant vapor from the catheter under vacuum, and for controlling a compressor to pressurize the coolant vapor into a liquid stored in a recovery tank. In addition to the liquid refrigerant, a secondary heat removal or dissipation element, such as a conductive coil may be used.

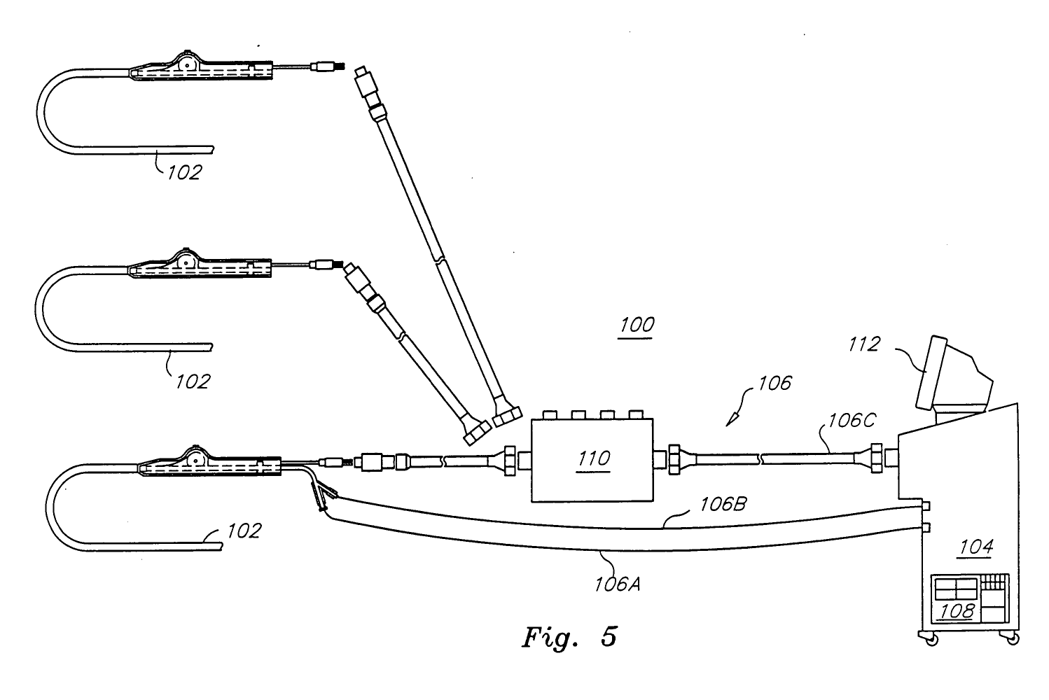

The multi-energy ablation station 100 produces controlled temperatures at the tip of a catheter 102. A selected one or more catheters may be coupled to the console 104 (as shown in FIG. 5). The catheters 102 are typically long, flexible catheters that can be inserted through various body passages. Various types of catheters, including balloon catheters, or even probes, may be used. The present invention is compatible with catheters or probes that are equally adaptable for both endovascular and surgical procedures. Because system 100 is capable of supplying more than one type of energy to the catheters, the preferred embodiment of the invention provides catheters that are equally adaptable for RF ablation, cold-tipped RF ablation, cryoablation and cryomapping procedures.

Catheter 102 may be a focal-tip catheter that produces a concentrated zone of tissue destruction (ablation), or a linear catheter that delivers cold along the length of a catheter. Catheter tip structures that are adaptable with the present invention are described in U.S. Pat. Nos. 6,468,268 and 5,899,899, incorporated herein by reference. The user may wish to ablate the target tissue via a cryoablation procedure, commonly known in the art. In this instance, cryogenic fluid may be delivered to the catheters. Cryogenic fluid may comprise a combination of various gases and liquids including but not limited to argon, carbon dioxide, nitrous oxide, liquid nitrogen or the like. If pressurized gas is delivered, the gas is allowed to expand in the catheter tip creating cooling via the Joule-Thompson effect. As the pressurized gas expands rapidly inside the catheter cooling area, a thermally conductive segment on the catheter is chilled which leads to a chilling of the target tissue.

In the embodiment shown in FIG. 1, a radiofrequency generator 108 is coupled to an electrocardiogram (ECG) connector box 110. A user may select the type of ablation procedure he or she wishes via controls in the console 104. Catheter 102 may include one or more electrodes around its periphery. If the user wishes to perform an RF ablation procedure, radiofrequency energy can be provided to the electrodes of catheter 102 via electrical umbilical 106 to perform an RF ablation technique as is common in the art. RF energy is provided between electrodes situated on or within catheter 102 and a collector/ground plate 109. Plate 109, is typically positioned on the patient's body. RF energy is caused to flow within the patient's tissue between the electrode and collector/ground plate 22, treating the targeted tissue.

Instead of or concurrent with the delivery of RF current, a conductive refrigerant may be delivered to the catheter to provide the electrical connection to the catheter's electrodes. Microwave energy or direct current may also be supplied to the catheter 102. Further, laser ablation may be performed by passing an optical fiber through the electrical umbilical 106C. Light energy generated from within console 104 is passed through the optical fiber to the catheter 102 to perform laser ablation as is commonly known in the art.

The present invention allows for various combinations of different types of ablation energy to be delivered to one or more catheters, which ablate the target tissue region. The ablation procedures can take place in a sequential manner, depending upon the severity of the tissue region being treated. The ability to quickly switch from one ablation procedure to another may provide improved treatment of tissue lesions. For example, a surgeon may cryoablate a tissue region for 60 seconds until a local edema is created. RF ablation may then follow after water has accumulated proximate the tissue region to allow RF energy to more easily spread deeper into the tissue region.

In another scenario, a target tissue region is cryomapped prior to cold-tip RF ablation. For this purpose, the user would preset a fixed RF power, for example, 80 Watts, on generator 108 and initiate controller 104 to control and maintain the tip temperature to a predefined temperature value, for example +20° C. While RF energy is flowing through the tip to the tissue, the tip temperature would rise to very high temperature, which creates tissue burning. In order to prevent such a phenomena, the controller, via console 104, delivers the cryogenic fluid which lowers, controls and maintains the tip temperature at a colder value, for example around +20° C. The tissue inside the targeted area will still be destroyed by the RF energy due to the high temperatures but the surface of the heart tissue will not be destroyed due to the delivery of the cryogenic fluid.

In another embodiment, the user again presets a fixed RF power on generator 108 and initiates console 104 to control the tip temperature. However, in this scenario, in order to maintain the tip temperature at a defined value, the user, via console 104, applies a controlled range of temperature values, i.e. ramping the temperature values in order to maintain tip temperature at a predefined value. Therefore, a final tip temperature is again achieved, but it is achieved via an incremental lowering of the temperature by controlling the amount of cryogenic fluid delivered to the catheter. In this embodiment, a final temperature is again reached, but it is reached via a controlled release of cryogenic fluid by applying incremental temperature settings via console 104.

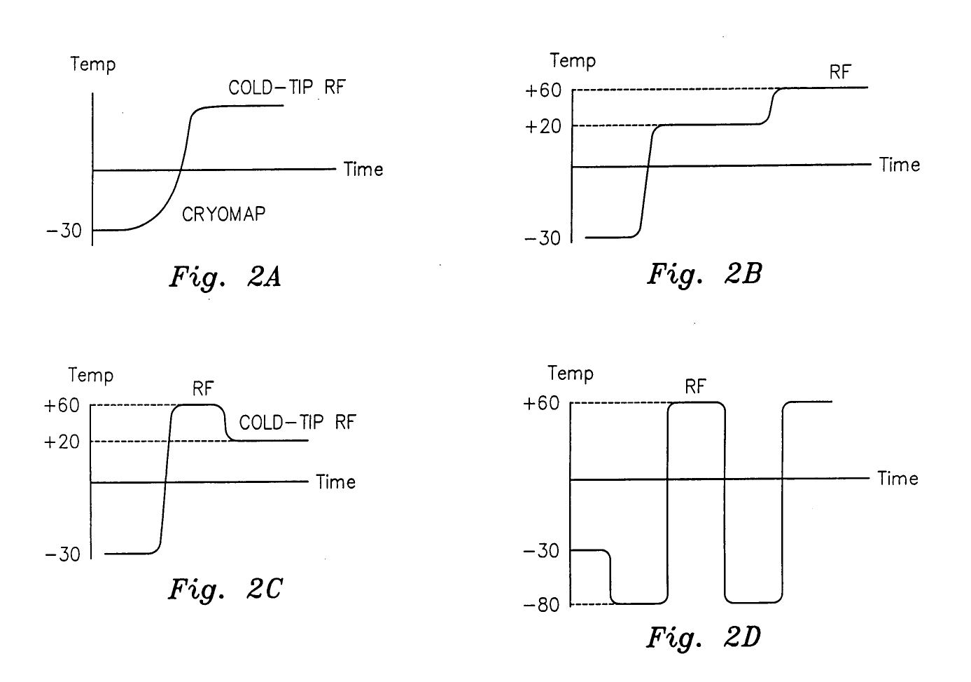

The console 104 can be used to control the tip temperature through predefined profiles. The controller could control the tip temperature during an ablation procedure by any one of a number of different profiles, including, but not limited to those shown in FIGS. 2A-2D. In FIG. 2A, an exemplary ablation/temperature profile utilizing the present invention is illustrated. Here, cryomapping is initially performed at very low temperatures for a certain period of time and RF energy is then delivered via the catheter, raising the tip temperature. As described above, in order to maintain a specific temperature range, cold-tip RF ablation is performed by delivering cryogenic fluid along with RF energy to the tissue to maintain the tip temperature within a certain range.

FIG. 2B illustrates a different temperature profile. Here, ablation is performed at 30° C. for a period of time, followed by RF ablation, which increases the temperature from, in this example, +20° C. to +60° C.

FIG. 2C illustrates yet another exemplary profile where cryomapping is performed prior to RF ablation. Cryomapping is performed at or about −30° C. RF ablation brings the temperature of the catheter tip up to approximately +60° C. At this point, cryogenic fluid is released and delivered to perform cold-tip RF ablation via the above-described procedure, which lowers the catheter tip temperature from approximately +60° C. to approximately +20° C.

Finally, in FIG. 2D, in yet another exemplary embodiment, cryoablation and/or cryomapping may be performed at very low temperatures, i.e. between −30° C. and −80° C., at which point RF ablation is performed. A cycling of ablation procedures may then occur, varying the temperature between −80° C. and +60° C. The embodiments in FIGS. 2A-2D are exemplary and illustrate only a few of the many temperature/ablation profiles that can be formed using the variety of ablation and mapping procedures capable of being sequentially performed using the present invention.

The cryoablation/radiofrequency ablation system 100 of the present invention allows the user to select an appropriate ablation procedure depending upon the patient's need. Monitor 112 is coupled to a processor inside console 104 enabling the user to monitor and control the ablation procedures, adjusting the amount of RF, microwave or direct current, or cryogenic fluid that is supplied to catheter 102. The user, by selecting and combining different ablation procedures can cover a wide temperature spectrum and effectively treat different types of lesions.

Prior to an RF ablation procedure, the tissue containing the lesion may be mapped via a standard cryomapping (ice mapping) procedure. In one instance, for example, the cooled tip of catheter 102 is placed at the proposed lesion site. When the cardiac tissue reaches approximately +5 degrees Celsius, its electrical activity is suppressed. If the proposed lesion site can be therapeutically effective when ablated, a condition such as arrhythmia will no longer be inducible once the electrical activity of the proposed site is suppressed by cooling. Having confirmed the effectiveness of the proposed site, ablation via any of the ablation techniques described above is performed in manner known to those skilled in the art. The process of cryomapping followed by RF, cold-tip RF, cryo or any other form of ablation may be performed quickly and efficiently without exchanging one catheter for another.

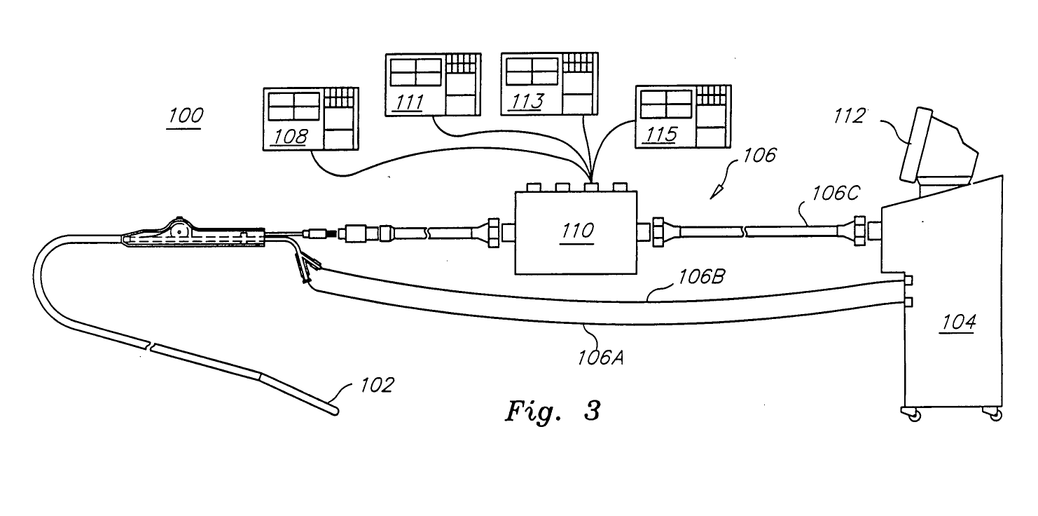

FIG. 3 illustrates another embodiment of the present invention. This embodiment shows the capability of the ablation station 100 to couple to any one of a variety of different commercial generators. In FIG. 3, an RF generator 108, microwave generator 111, ultrasound generator 113, or laser light generator 115, may be coupled to ECG connector box 110. In this embodiment, catheter 102, a cryoablation catheter, is designed to deliver one or more energy types.

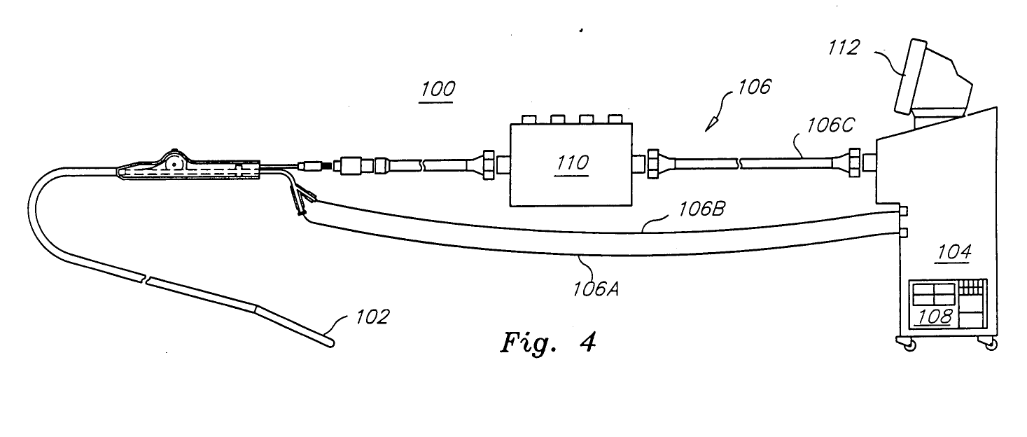

FIG. 4 depicts yet another embodiment of the multi-energy ablation station 100 of the present invention. In this exemplary embodiment, an RF generator 108, or a generator delivering other types of energy, such as cryogenic fluid, is included within console 104. In this fashion, a fully integrated multi-energy ablation station is provided, wherein combinations of different ablation procedures may be performed. For example, cryomapping of the target tissue are may be performed prior to cryoablation, RF ablation, or cool-tip RF ablation. A different type of energy generator may be used in lieu of the RF generator in console 104, such as, for example, a microwave generator.

FIG. 5 depicts yet another embodiment. Station 100 is adaptable to be coupled to one of a plurality of multi-functional catheters 102. The catheter 102 may be enabled for RF ablation, RF cool-tip ablation, cryoablation, cryoadhesion, and/or cryomapping procedures. In the preferred embodiment, console 104 includes an RF generator 108, which supplies radiofrequency energy to one or more catheters 102 via electrical umbilical 106C. As described above, other forms of electrical energy in the form of dc current or microwave energy maybe provided through electrical umbilical 106 by substituting various types of generators within console 104. Laser energy may also be provided via an optical fiber embedded within electrical umbilical 106.

In the preferred embodiment, catheters 102 can be made up of any commercial RF ablation or cryoablation catheters, as well as a hybrid type of catheter having the ability to ablate tissue via either RF or cryoablation techniques. A processor in the console allows a user to selectively control which catheter will receive energy, and which type of energy it will receive. A user may, for example, choose to treat the target tissue with a conventional cryoablation procedure. A cryogenic fluid may be supplied to a conventional cryocatheter via the injection umbilical. Cryomapping can take place prior to the cryoablation using conventional mapping techniques.

The user may, instead, choose to treat the target tissue using an RF ablation procedure. The processor in the console is then directed to access an RF-capable catheter already coupled to the system via the electrical umbilical 106C. Direct current may also be sent in lieu of RF energy. The RF catheter now receives RF energy and treats the tissue via conventional RF ablation techniques. Prior to the lesions created by the RF ablation, cryomappng can be performed first. This would be accomplished by first enabling a cryocatheter to map the target area. Any combination of ablation procedures can treat the target tissue by first enabling a compatible cryo-treatment catheter or RF-treatment catheter. In this fashion, a wide range of temperature ranges can be achieved without the need to change and/or replace one type of catheter with another.

Virtually any combination of ablation procedures can be performed on a target tissue area. For example, cryoablation may be performed with or without a cryomapping procedure preceding it. Conversely, RF ablation may be performed with or without cryomapping prior to the procedure. A sequence of ablation procedures may be performed. For example, RF ablation followed by cryoablation, or vice-versa. Cryoablation or RF ablation, followed by cold-tip RF ablation is yet another sequence. If a fluid-cooled cold-tip RF ablation procedure is desired, RF energy is supplied to one of the catheters 102 capable of receiving both RF energy and cryogenic fluid. RF energy is supplied to the electrodes in the catheter 102, while a controlled release of coolant passes through the lumens in the catheter to cool down the ablation area.

A cycling mode can be implemented wherein a sequence of ablation procedures can be programmed and controlled by the processor. Catheters 102 can be comprised of RF catheters, cryocatheters, or catheters with the ability to ablate via either technique. The user provides input to a computer processor within console 104. These inputs provide activation signals to the catheter, and control the release of cryogenic fluid and/or radiofrequency energy through umbilical system 106 to the catheter 102.

Simultaneous ablation is also a feature of the present invention. Different types and quantities of ablation energies may be delivered to the one or more catheters 102 to perform simultaneous ablation procedures. For example, in a multi-catheter scenario, one catheter 102 may receive coolant for a cryoablation procedure, while RF energy may be supplied to another catheter via the RF generator 108 and the electrical umbilical 106C. Cryoadhesion, a procedure where the tip of the catheter adheres to the tissue to reduce the risk of accidental slippage of the catheter tip, may also be performed, followed or preceded by any of standard ablation techniques.

The cryoadhesion procedure described above may be performed simultaneously with the delivery of a second type of energy, such as, but not limited to, RF, microwave and laser light and ultrasound energies. For example, RF energy can be delivered to catheter 102 while the catheter is performing a cryoadhesion procedure. In this scenario, the catheter tip is comprised, preferably, of two, co-centered electrodes. One of the electrodes is to deliver RF energy and the other is for freezing or cooling the surrounding tissue. When the ablation system of the present invention operates in this fashion, the system supplies RF energy to the lesion site while the catheter tip performs cryoadhesion in order to reduce the likelihood of slippage of the catheter tip. Since the impedence of ice is higher than that of tissue, the RF energy travels undiffused into the tissue, creating a deeper narrower lesion than would otherwise occur. The ablation system can operate as a cooled-tip RF system where the ice cools the tissue directly, as opposed to prior existing systems that only cool the ablation electrode.

The present invention is not limited to a specific number of catheters, or a specific sequence of ablation sequences. The multi-energy ablation station of the present invention allows for a variety of different types of probes or catheters, each enabled to perform one or more ablation procedures, coupled to an umbilical system that selectively supplies various types of energy necessary for mapping and ablating an area of the body.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

1. An ablation station having multiple energy treatment capabilities for performing sequential or simultaneous ablation techniques to a target tissue area, the ablation station comprising:
a treatment energy generation station capable of supplying one or more different forms and levels of treatment energy to one or more energy treatment devices; and
an umbilical system having a first end adaptable for coupling to the one or more energy treatment devices and a second end coupled to the energy generation station,
wherein the treatment energy generation station comprises a processor that can selectively disperse the one or more different forms and levels of treatment energy and selectively activate the one or more energy treatment devices.

2. The ablation station of claim 1 wherein the one or more energy treatment devices are probes.

3. The ablation station of claim 1 wherein the one or more energy treatment devices are catheters.

4. The ablation station of claim 3 wherein the one or more energy treatment devices are balloon catheters.

5. The ablation station of claim 3 wherein the one or more catheters are cryocatheters.

6. The ablation station of claim 3 wherein the one or more catheters are linear catheters.

7. The ablation station of claim 3 wherein the one or more catheters are radiofrequency catheters.

8. The ablation station of claim 1 wherein the one of the one or more energy treatment devices are capable of both cryoablation and radiofrequency ablation.

9. The ablation station of claim 1 wherein the treatment energy generation station includes a radiofrequency signal generator that supplies radiofrequency energy to the one or more energy treatment devices.

10. The ablation station of claim 1 wherein the energy generation station supplies cryogenic fluid to the one or more energy treatment devices.

11. The ablation station of claim 10 wherein the energy generation station selectively supplies the cryogenic fluid and a second type of energy to the one or more energy treatment devices.

12. The ablation station of claim 11 wherein the second type of energy is radiofrequency energy.

13. The ablation station of claim 11 wherein the second type of energy is microwave energy.

14. The ablation station of claim 11 wherein the second type of energy is ultrasound energy.

15. The ablation station of claim 11 wherein the second type of energy is laser light energy.

16. The ablation station of claim 12 wherein the energy generation station simultaneously supplies the radiofrequency energy and the cryogenic fluid to the one or more energy treatment devices.

17. The ablation station of claim 1 wherein the energy generation station supplies microwave energy to the one or more energy treatment devices.

18. The ablation station of claim 1 wherein the energy generation station supplies laser light energy to the one or more energy treatment devices.

19. The ablation station of claim 18 wherein the umbilical system includes a fiber optic cable coupling the energy generation system to the one or more energy treatment devices.

20. The ablation station of claim 1 wherein only cryoablation procedures are performed.

21. The ablation station of claim 20 wherein cryomapping of the target tissue area is performed prior to the cryoablation procedures.

22. The ablation station of claim 1 wherein only radiofrequency ablation procedures are performed.

23. The ablation station of claim 22 wherein cryomapping of the target tissue area is performed prior to the radiofrequency procedure.

24. The ablation station of claim 1 wherein a sequence of ablation procedures are performed, the sequence comprising radiofrequency ablation followed by cryoablation.

25. The ablation station of claim 1 wherein a sequence of ablation procedures are performed, the sequence comprising cryoablation followed by radiofrequency ablation.

26. The ablation station of claim 1 wherein a sequence of ablation procedures are performed, the sequence comprising cryoablation followed by cold tipped radiofrequency ablation.

27. The ablation station of claim 1 wherein a sequence of ablation procedures are performed, the sequence comprising radiofrequency ablation followed by cold tipped radiofrequency ablation.

28. The ablation station of claim 1 wherein the processor allows for selective cycling between the one or more different forms and levels of treatment energies.

29-56. (canceled)
